(19) United States
(12) Patent Application Publication
(10) Pub. No.: US 2007/0121065 A1
(43) Pub. Date: May 31, 2007

(54) Device and method for tracking eye gaze direction

(76) Inventors: David D. Cox, Somerville, MA (US); James J. DiCarlo, Boston, MA (US)

Correspondence Address: PEARL COHEN ZEDEK LATZER, LLP, 1500 BROADWAY 12TH FLOOR, NEW YORK, NY, 10036, US

(21) Appl. No.: 11/386,878

(22) Filed: Mar. 23, 2006

Related U.S. Application Data
(60) Provisional application No. 60/664,593, filed on Mar. 24, 2005.
(60) Provisional application No. 60/667,682, filed on Apr. 4, 2005.

(51) Int. Cl. A61B 3/14

(52) U.S. Cl. 351/209

(57) ABSTRACT

Eye-tracking devices and method of operation may utilize a magnetic article associated with an eye and a sensing device to detect a magnetic field generated by the magnetic article.

### CROSS REFERENCE TO RELATED APPLICATIONS

This application claims the benefit of U.S. Application Ser. No. 60/664,593, filed on Mar. 24, 2005, and U.S. Application Ser. No. 60/667,682, filed on Apr. 4, 2005, both of which are incorporated in their entirety herein by reference.

### GOVERNMENT INTEREST STATEMENT

This invention was made in whole or in part with government support under funding by the Office of Naval Research Contract No. F49620-02-c-0041 for the National Defense Science and Engineering Graduate Fellowship (NDSEG); Contract No. 5-RO1-EY014970-02, “Visual Object Processing in the Inferotemporal Cortex”, for NIH NEI RO1; Contract No. N00014-02-1-0915, “A Cortical Interface for the Analysis and Synthesis of Object Recognition” for DARPA/ONR; and Contract No. 5-P20-MH66239-03, “Detection and Recognition of Objects in Visual Cortex” for NIH (Conte Center Grant). The government may have certain rights in the invention.

### BACKGROUND OF THE INVENTION

1. Field of the Invention

The present invention generally relates to eye-tracking devices. Specifically, the present invention relates to devices which may include a magnetic article associated with the eye of a user. In particular, the device may include a sensing device for detecting a magnetic field generated by the magnetic article. More specifically, the device may include tracking eye movements by a generated magnetic field and transmitting this data to a processor. By tracking eye movement with the device of the present invention, precision guidance of articles controlled by a processor can be accomplished with heretofore unrealized results. More particularly, a device is disclosed including instructing an assistive device, a computer or a machine, and methods for tracking the movement or position of an eye.

2. Description of the Related Art articles. Currently, devices for accurately tracking eye movement, for example, devices suitable for use with humans, are needed for many application, including diagnosis and treatment of vision and eye-movement related medical disorders, assistive devices for, e.g, challenged or handicapped persons, computer-generated animation, and various military applications.

Accordingly, there is now provided with this invention an improved eye tracking device which overcomes longstanding problems inherent in the art. These problems have been solved in a simple, convenient, and highly effective way for tracking eye movements and using this data for providing guidance instructions.

### SUMMARY OF THE INVENTION

According to one aspect of the invention, an eye-tracking device for tracking movement or position of an eye of a subject is disclosed. Such a device includes a magnetic article adapted for association with said eye and a sensing device for detecting the magnetic field of said magnetic article.

Another aspect of the present invention includes a method for tracking a movement or position of an eye of a subject. The method includes detecting a magnetic field with a sensing device, associating a change in the magnetic field with movement of the eye, and transmitting data representing said eye movement.

A still further aspect of the present invention includes a method for controlling a machine by eye movement. Such a method includes detecting a changing magnetic field by a sensing device associated with an eye, creating data representing the changing magnetic field, and transmitting data representing said eye movement. The method further includes controlling said machine by said transmitted data.

As will be appreciated by those persons skilled in the art, a major advantage provided by the present invention is the control of a wide variety of machines, device, and appurtenances by means of eye movement. It is therefore an object of the invention to detect the movement of an eye and control a device by such movement. Additional objects of the present invention will be better understood by reference to the following detailed discussion of the invention and the attached figures which illustrate specific embodiments.

### BRIEF DESCRIPTION OF THE DRAWINGS

Embodiments of the present invention are described with reference to the following drawings, wherein:

FIG. 1A is a schematic diagram of an exemplary eye-tracking device, according to an embodiment of the present invention.

FIG. 1B is a diagram of the electronics of an embodiment of the present invention.

FIG. 1C is an orthogonal abstract diagram of the electronics of an embodiment of the present invention.

FIG. 1D is an orthogonal abstract diagram of the electronics of an embodiment of the present invention.

FIG. 2 is a generalized schematic diagram of the sensor assembly of an embodiment of the present invention.

FIG. 3 depicts a circuit diagram of the sensor portion of an embodiment of the present invention.

FIGS. 4A-C depict alternative arrangements of magnetic elements and magnetic particles in a contact lens of an embodiment of the present invention.

FIGS. 5A-G depict alternative arrangements of sensors affixed to a pair of eye glasses of an embodiment of the present invention.

FIGS. 6A-C depict computer-simulated flux measurements from a device containing differential sensors in three dimensions, according to an embodiment of the present invention.

FIGS. 7A-C depict a differential outputs of a pair of sensors as a function of azimuth and elevation angles of a goniometer.

FIG. 8 is a subset of the measurements shown in FIGS. 7A-C and depicts the sensor outputs as a function of the azimuth and elevation angles of a goniometer.

FIGS. 9A-B depict the surgical procedure followed to implant a magnet beneath the conjunctiva of an eye.

FIGS. 10A-D depict the output of a pair of sensors of an embodiment of the invention attached to a non-human primate, as compared to an existing eye-tracking system.

FIGS. 11A-B depict a magnified view of measurements illustrated in a detail 1 of FIGS. 10A and 10C.

FIGS. 11C-D depict a magnified view of measurements illustrated in a detail 2 of FIGS. 10A and 10C

FIGS. 12A-B depict the construct of a contact lens with a magnet attached to it.

FIGS. 13A-B depict data collected from two sensor channels of an embodiment of the present invention.

FIGS. 14A-B depict eye positions estimated using a video eyetracker.

FIGS. 14C-D depict eye positions estimated using an embodiment of the present invention simultaneously with that of FIGS. 14A-B.

### DETAILED DESCRIPTION OF THE INVENTION

In the following detailed description, numerous specific details are set forth in order to provide a thorough understanding of the invention. However, it will be understood by those of ordinary skill in the art that the present invention may be practiced without these specific details. In other instances, well-known methods, procedures, components, and structures may not have been described in detail so as not to obscure the present invention.

Embodiments of the invention provide, for example, eye-tracking devices, an apparatus for instructing an assistive device, a computer or a machine. Embodiments also include and methods for tracking movement or position of an eye and for instructing an assistive device, a computer or a machine. Embodiments further include a magnetic article associated with an eye, and a sensing device for detecting a magnetic field generated by the magnetic article.

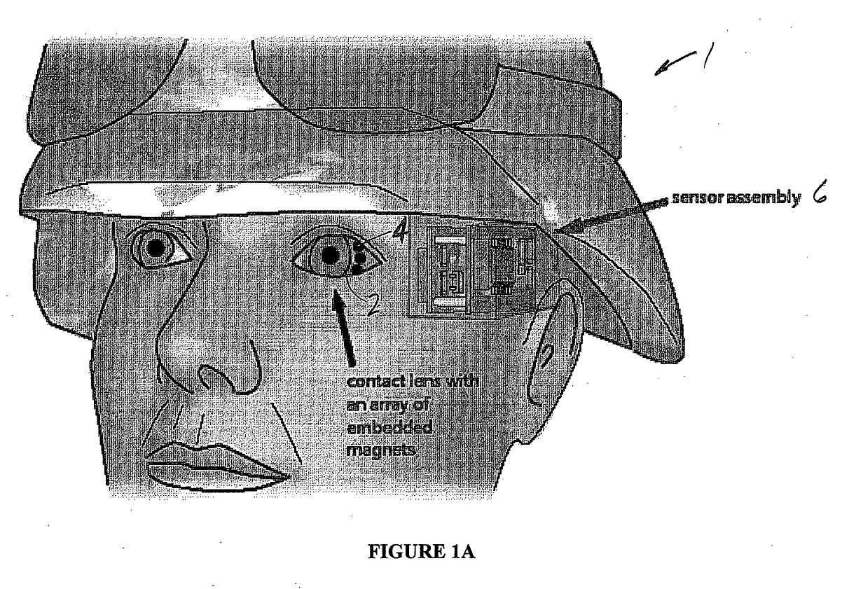

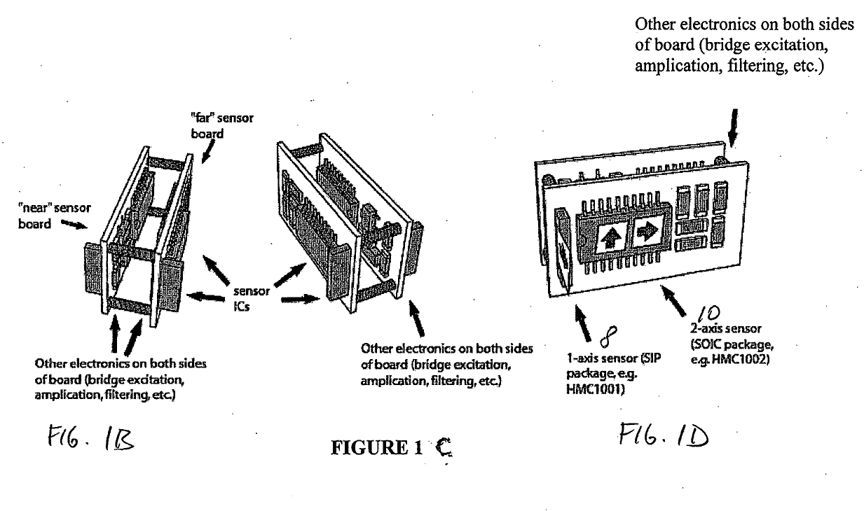

FIG. 1A depicts a schematic diagram of an exemplary eye-tracking device. In one embodiment, the device can be used, for example, for military applications; e.g, for aiming guns, grenade launchers, rocket launchers, mortars, machine guns, sub-machine guns, rifles, handguns, pistols, bows, anti-aircraft weapons, anti-ballistic devices, anti-tank weapons, anti-personnel weapons, ballistic missiles, etc. In other embodiments, an eye tracking device can be used for other applications, such as controlling or providing input to computers or software.

As shown in FIG. 1A, a soldier 1 is wearing a contact lens 2 having an array of magnetic elements 4 embedded in a radial fashion in the contact lens. A single magnetic article could also, of course, be used. In one embodiment, the magnetic elements are fully embedded in the lens and do not touch the eye. The placement of multiple separate elements in the lens increases the total magnetic flux emanating from the lens while allowing the lens to remain flexible. As shown in FIGS. 1B-1D, attached to the side of the soldier's head is a sensor assembly 6, preferably containing two 1-axis sensors 8 and two 2-axis sensors 10. This sensor assembly enables accurate determination of the position of the magnetic device and therefore of the soldier's gaze in all three dimensions.

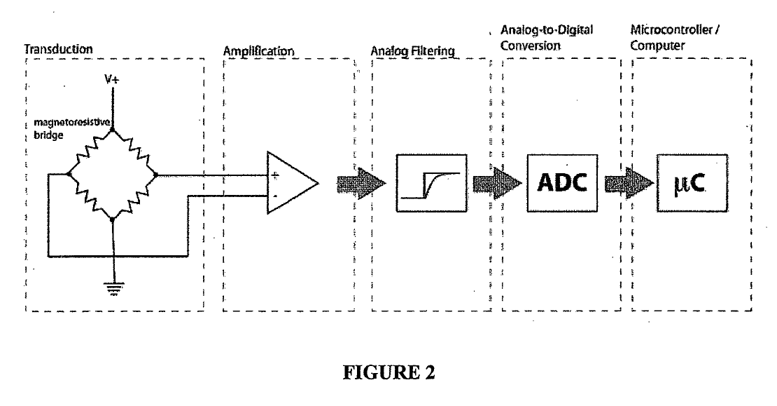

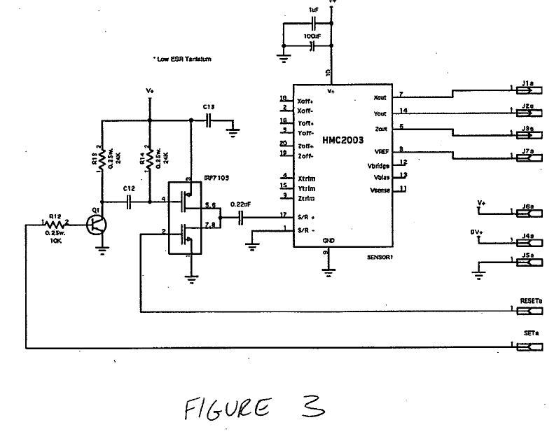

As illustrated in FIG. 2, an embodiment of the present invention includes signal transduction, amplification, filtering and/or analog-to digital conversion electronics. Such electronic components are each well known in the art. Specifically, FIG. 2 depicts the components typically associated with a single sensor channel, according to one embodiment of the invention. The microcontroller and/or computer would be responsible for translating sensor outputs into eye position estimates, using any of a variety of algorithms described below. Digital filtering could additionally be performed by a digital signal processor (DSP) subsequent to analog-to-digital conversion, or could be performed by the microcontroller/computer. While FIG. 2 shows a magnetoresistive sensor arranged in a Wheatstone bridge, any magnetic sensing circuit known in the art could be used.

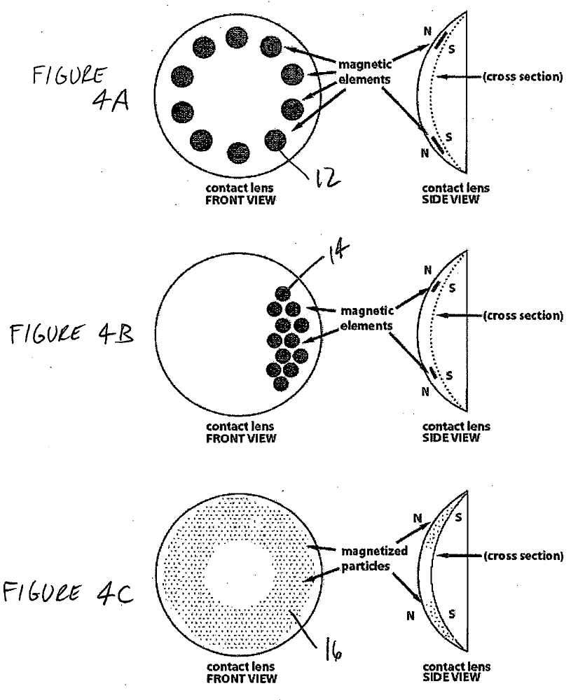

FIG. 3 depicts a circuit diagram of an embodiment of the present invention, covering the transduction, amplification, and analog filtering of the signal (first three boxes in FIG. 2). In one embodiment of the invention, two such circuits were constructed and their outputs were fed into a National Instruments USB6009 analog-digital convertor, which was in turn, attached to a personal computer. Sensor measurements were recorded to disk, and were analyzed using Matlab (Mathworks, Inc. Natick, Mass.) to produce the data.

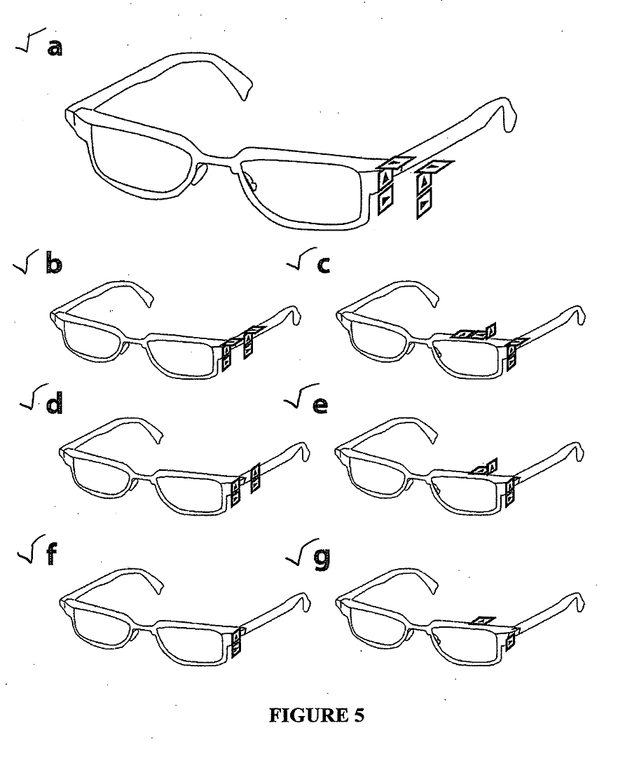

FIGS. 4A-C depict some alternative arrangements of magnetic elements and magnetic particles in the contact lens. Specifically, FIG. 4A illustrates a radially symmetric arrangement of magnetic particles distributed evenly 12 around the circumference of the lens. FIG. 4B depicts another arrangement of magnetic elements in a contact lens. As shown, the magnetic elements are packed more densely on a side portion 14 of the lens. In this embodiment, the magnetic elements are located only on the side closest to the sensor array. In a further embodiment shown in FIG. 4C, the magnetic elements are widely distributed 16 throughout the lens, provided that they do not block the soldier's vision. In this embodiment, the contact lens has the magnetized particles embedded therein. This embodiment may be manufactured by having unmagnetized particles embedded in the lens during manufacture, which are later magnetized by a large external magnetic field. Of course, other methods of manufacturing a lens having widely distributed magnetized particles may be used for creating a lens depicted as shown in FIG. 4C. Different distributions of magnetic material typically produce different flux vector fields, resulting in different systematic relationships between sensor measurements and eye positions. The methods for translating sensor outputs to eye position estimates described below do not depend on the exact arrangement of magnetic material associated with the eye, and thus a wide range of arrangements may be used. In general, embodiments that produce larger magnetic fields (more, stronger magnetic material) are preferred, as they will produce the largest variation in sensor output as the eye moves.

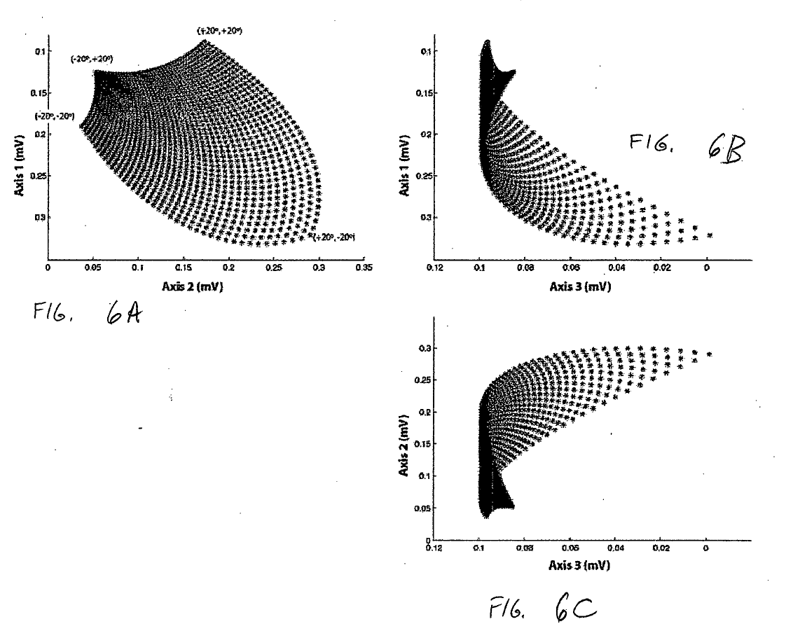

FIGS. 5a-5g depicts a variety of arrangements of sensors affixed to a pair of eye glasses. In other embodiments, similar arrangements of sensors may be coupled to the head of a user in other ways, for example, on a head band. In the arrangements depicted, more than one array of sensors may be used. In this way, various kinds of differential measurements can be made. In other embodiments, the measurement or tracking of eye position may be performed without using multiple arrays. In the variety of arrangements shown in FIGS. 5a-g, sensors are typically shown schematicallyas squares with an arrow indicating their direction of sensitivity. For example, FIG. 5a depicts a sensor arrangement with a near x-y-z and far x-y-z array. This arrangement is also depicted in FIG. 1A. As in FIG. 1A, this sensor geometry is consistent with that of the Honeywell HMC2003 3-axis sensor. FIG. 5b depicts an arrangement where the near and far arrays are arranged differently. FIG. 5c depicts an arrangement wherein differential measurements are made across two x-y-z arrays in a still different configuration. FIG. 5d depicts an arrangement wherein the sensors in each array do not span all three x-y-z directions. FIG. 5e depicts an arrangement wherein two arrays of sensors span the same space of directions, but not in exactly the same configuration. FIG. 5f depicts a further arrangement with two sensors which are not arranged in differential pairs. FIG. 5g depicts another two-sensor arrangement. Any of the above-described arrangements (and others) could be used to estimate eye position. Naturally, different arrangements of sensors may typically require different functions to translate sensor outputs into eye position estimates. However, embodiments of the methods for computing translation functions described below would apply equally well to any arrangement.

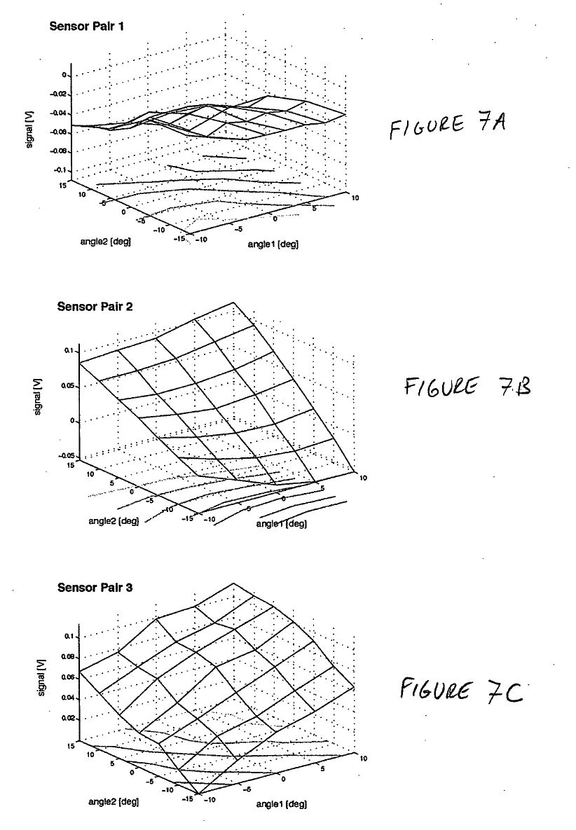

FIGS. 6A-6C illustrate the results of a computer-simulated test of another embodiment of the present invention. An eye-tracking device similar to that depicted in FIG. 1 was simulated over a range of +/−20 degrees movement, in 1-degree increments. Axis 1 represents the output of the proximal x-axis sensor minus the output of the distal x-axis sensor; axis 2 and axis 3 similarly, are the differences between the outputs of the y-axis sensor and z-axis sensor, respectively. The simulated magnet was a 1 mm thick, 2 mm diameter NdFeB (N42) cylinder, and was modeled as a simple magnetic dipole (which provides a good approximation of the far-field characteristics of a cylindrical permanent magnet). Computer-simulated flux measurements are depicted in FIG. 6.

In other embodiments, the devices and methods of the present invention maytrack eye movement or eye position over wide range of angles, typically over a range of about +/−4 degrees, a range of about +/−6 degrees, a range of about +/−8 degrees, a range of about +/−10 degrees, a range of about +/−12 degrees, a range of about +/−14 degrees, a range of about +/−16 degrees, a range of about +/−18 degrees, a range of about +/−22 degrees, a range of about +/−25 degrees, a range of about +/−30 degrees, a range of about +/−35 degrees, a range of about +/−40 degrees, a range of about +/−50 degrees, or a range of about +/−60 degrees.

The magnetic article of an embodiment of the present invention may comprise a wide choice of materials, for example, any ferromagnetic material, including neodymium-iron-boron, FeCoB, FeCoNiB, an alloy material comprising iron, nickel and/or cobalt, at least one element selected from the group consisting of Fe (iron), Co (cobalt), Ni (nickel), Ti (titanium), Zn (zinc), Cr (chrome), V (vanadium), Mn (manganese), Sc (scandium), and Cu (copper). Neodymium-iron-boron alloys are preferred as they generally produce the strongest magnetic fields.

In another embodiment of the invention, the article associated with the eye may not itself generate a magnetic field, but rather, may distort an external magnetic field in the vicinity of the article associated with the eye. This article may comprise a wide range of materials of magnetic permeabilities ranging from a high magnetic permeability to a medium magnetic permeability. The article in one such embodiment preferably comprises Permalloy® Fe—Ni—Mo, because it exhibits very high magnetic permeability. In one embodiment, the magnetic permeability of the magnetic article may create a distortion in an external magnetic field that can be measured by a sensor of the present invention. Another embodiment of the present invention provides devices, apparatuses, and methods in which an article that may distort an exogenous magnetic field (rather than generating a field itself) is associated with the eye.

In another embodiment, the magnetic article may comprise a material that exhibits super paramagnetism. In another embodiment, the magnetic article may comprise iron oxide nanoparticles. In another embodiment, the magnetic article comprises any other magnetic material known in the art.

The magnetic article of an embodiment of the present invention may be attached to or associated with different parts of the eye. For example, the magnetic article may be attached to the conjunctiva of the eye, the sclera, between the sclera and the conjunctiva, or to any other part of the eye. The magnetic article may be also associated with or attached to a contact lens, or to any other device associated in any way with the eye.

The sensing device of an embodiment of the present invention responds generally to spatial differences of the magnetic field formed by the magnetic article. Exemplary magnetic sensing devices are depicted in FIGS. 2 and 3. Other examples of magnetic sensing devices may be any of the following Honeywell sensors: HMC1001, HMC1002, HMC1021S, HMC1021Z, HMC1021D, HMC1022, HMC1023, HMC1041Z, HMC1051Z, HMC1051ZL, HMC1052, HMC1052L, HMC1053, HMC1055, HMC1501, HMC1512, HMC6352, and HMC2003; and any of the following NVE Corporation sensors: the AA-Series, AAH-Series, and AAL-Series GMR Magnetometer sensors and the AB-Series and ABH-Series GMR Gradiometer sensors, or any other sensing device known in the art. Other examples include magnetoresistive sensors based on the spin-dependent tunneling junctions and sensors that measure the Hall Effect produced by a magnetic field (e.g. Honeywell SS4, SS5, SS400 and SS500). Each sensing device represents a separate embodiment of the present invention. The use of magnetic sensing devices is well known in the art, and can be obtained, for example, from the literature accompanying any of the above sensors.

Generally, the sensing device of an embodiment of the present invention may be attached in some way to the head of the subject or user. Preferably, the sensing device may be attached to the side of the head so as not to obstruct the view of the subject or user. Of course, the sensing device may be attached to any part of the body or article associated with any part of the body. For example, the sensing device may be attached to a helmet, eyeglasses, goggles, backpack, belt, etc. Preferred embodiments of the invention would place the sensors close to the eye. One object or element may be attached to another via an adhesive material, a wire, cable, string, a suture or any other mechanism of attachment known in the art. As used herein, “attached to” refers to an association between two objects or elements, either direct or indirect.

In another embodiment, one object or element may be embedded in another; e.g. a magnetic article may be embedded in a conjunctiva, a sclera, a contact lens, or the like.

In another embodiment, the sensing device may include a differential sensor. The differential sensor of an embodiment of the present invention may further include a plurality of single-axis sensors, the single-axis sensors having identical or similar orientations. A “differential sensor” is typically one that computes the difference in magnetic fields detected by two like-oriented sensors, here referred to as a “differential pair” of sensors. However, the differential sensor may also be any other type of differential sensor known in the art. To the extent that sensors in a “differential pair” are oriented in similar directions, the impact of interfering magnetic sources on the measured signals may be more similar (particularly if the interfering source is far away and the two sensors in the pair are close to each other). Thus, taking the difference between the outputs of sensors in a differential pair may be used to cancel-out some of the effect of interfering magnetic sources.

In one embodiment, the distance between the sensors in a differential pair and the magnetic article may be small relative to the distance between the pair and an interfering magnetic source (e.g. the Earth's magnetic field, a cathode-ray tube (CRT) monitor, or a building's power distribution). This enables an accurate calculation of the field generated by the magnetic article, because differences between the flux sensed by the sensors of the differential pair will typically be greater from the nearby (eye-associated) article, as compared with a distant interfering source. (This is typically because the strength of a magnetic field falls off as a function of the inverse cube of distance). The sensors of a differential pair of an embodiment of the present invention may be separated by a range from about 0.1 mm to about 2 mm and typically by about 1 mm. This separation may include distances of about 5 mm, 10 mm, and 15 mm. The closer the two sensors in a differential pair are, the more similarly they may be affected by a distant interfering source. For example, taking the difference between the outputs of the sensors in the pair will typically tend to cancel-out the effect of the interfering source. At the same time, however, if the sensors in a differential pair are close, they may also be affected more similarly by the flux generated by the magnetic article (the source-of-interest), so taking the difference of the two sensors will also tend to cancel-out the signal of interest The optimal distance between sensors in a differential pair typically ultimately depends upon the typical distances from interfering magnetic sources, and on the details of sensor sensitivities.

The orientation of a sensing device of an embodiment of the present invention generally refers to the axis of maximum sensitivity to detection of magnetic field strength. Orientations of elements may range from identical, one from another, to ones that are not detectably different, to those not significantly different. They may also be similar, referring generally to orientations differing by less than about 0.0001 degrees, less than about 0.0003 degrees, less than about 0.001 degrees, less than about 0.003 degrees, less than about 0.01 degrees, less than about 0.03 degrees, less than about 0.1 degrees, less than about 0.3 degrees, less than about 1 degree, or less than about 3 degrees.

The distance between two or more of the single-axis sensors of an embodiment of the present invention may be either approximately invariant or may vary. In one embodiment, the distances between sensors may be adjustable, and may also be tailored to a specific operating environment (e.g. to achieve better immunity to interfering sources) or to different physical constraints (e.g. to fit into a different helmet, or onto a differently sized head, etc.) “Approximately invariant” generally refers to no detectable fluctuation in the value or measurement of interest. However, there may be, in other embodiments, different fluctuations ranging from less than about 0.0001 percent, less than about 0.0003 percent, less than about 0.001 percent, less than about 0.003 percent, less than about 0.01 percent, less than about 0.03 percent, less than about 0.1 percent, less than about 0.3 percent, less than about 1 percent, or less than about 3 percent. “Approximately invariant” may also refer to fluctuations of less than about 0.0001 millimeter (mm), less than about 0.0003 mm, less than about 0.001 mm, less than about 0.003 mm, less than about 0.01 mm, less than about 0.03 mm, less than about 0.1 mm, less than about 0.3 mm, less than about 1 mm, or less than about 3 mm.

The sensing device of an embodiment of the present invention may include a magnetoresistive sensor which may include an anisotropic magnetoresistive (AMR), a “giant” magnetoresistive (GMR) sensor, a “colossal” magnetoresistive (CMR) sensor and/or a spin dependent tunneling (SDT) magnetoresistive sensor. It may be any type of magnetoresistive sensor known in the art. The magnetoresistive sensor preferably includes Permalloy® Fe-Ni-Mo, but may include any substance known in the art with magnetoresistive properties.

Another embodiment of the sensing device of the present invention may include an AMR sensor further including circuitry that can be used to reverse the direction of sensitivity of the AMR sensor to magnetic fields. In another embodiment, the AMR sensing device may include a polarizing magnet that can be used to reverse the direction of sensitivity of the AMR sensor to magnetic fields. Alternatively, a loop of wire may be used to reverse the direction of sensitivity of the AMR sensor may be contained in a die of an integrated chip. Any other mechanism known in the art of reversing the direction of sensitivity of the AMR sensor to magnetic fields may be used. The polarizing magnet for reversing the direction of sensitivity to magnetic fields of the AMR sensor of an embodiment of the present invention may be proximate to the sensing device, attached to the sensing device, within the shell of the sensing device, or separate from the sensing component or components thereof.

AMR sensors of a differential pair with similar orientations of an embodiment of the present invention may have the same sense of direction or an opposite sense of direction. Typically, the sensing device may include a differential sensor. The differential sensor may include a plurality of anisotropic magnetoresistive (AMR) multi-axis sensors, the multi-axis sensors may have identical or similar sets of orientations. The differential sensor may alternatively include one or more single-axis sensors and one or more multi-axis sensors.

Two or more orientations of each of the multi-axis sensors may be identical, similar, or a combination. For example, one or more orientations of each of the AMR multi-axis sensors of an embodiment of the present invention may be similar, while the other is identical, or one or more orientations of each of the AMR multi-axis sensors may be identical, while the other is similar. The distance between two or more of the AMR multi-axis sensors may be either approximately invariant or not.

The magnet of an embodiment of the present invention may be either an electromagnet, a permanent magnet, or any other type of magnet known in the art.

The sensors of the sensing device of an embodiment of the present invention may not be similarly oriented; rather, their directions of sensitivity may span the same linear subspace. For example, if a sensor array consisted of three or more sensing elements with unique orientations not all lying in one plane, then any other set of three or sensing elements with unique orientation not all lying in one plane could be used to make differential measurements with the first array. Similarly, if a sensor array consisted of two or more sensors with orientations all lying in one plane, then it could be used with another set of two or more sensors with orientations lying in the same (or approximately the same) plane could be used to make differential measurements. These examples illustrate just two possible examples of groups of sensors spanning the same linear subspaces.

The magnetic field generated by the magnetic article of an embodiment of the present invention maybe a magnetostatic field, an alternating field, a static field, a time-varying field, or any other type of magnetic field known in the art. In one embodiment, the alternating magnetic field may be generated using a microchip implant transmitter powered by an external dipole antenna as described in (McGary J, J App Clin Med Phys, 5(4):2945, 2004). The alternating magnetic field may be generated by any other means known in the art of generating an alternating magnetic field.

In one embodiment, the signal transmitted from the sensing device may reflect the movements of the magnetic article. In another embodiment, the signal transmitted from the sensing device may correspond with the movements of the magnetic article.

Sensor outputs of an embodiment of the present invention may be generally translated into eye positions by digitizing analog voltages using an appropriate analog-to-digital converter and translating these values into eye position values. These values include at least a horizontal angle and a vertical angle for describing the angular position of an eye in its socket. The position values may also include information about the distance in 3-dimensional space of the sensor array from the eye if the relationship between the sensor array and the eye is not rigidly fixed.

In a preferred embodiment, the system of an embodiment of the present invention may be calibrated before use by placing the eye in known positions, and recording the output of the sensors. When only horizontal and vertical angular values are sought, these values may all lie on a two-dimensional manifold in an ambient space having the same dimensionality as the number of sensor outputs considered. Calibrating the system generally may require fitting a function to the data on this manifold, by using any of a number of function fitting techniques well known in the art. Function fitting techniques may vary according to user's desires. When the data are approximately linear, as to a plane, or a polynomial surface, one technique may to fit the data using a linear least-squares fitting procedure. Of course, a more complex nonlinear function may be used to fit the data using one of various nonlinear optimization procedures as is well known in the art.

The magnetic fields produced by the magnetic elements of an embodiment of the present invention associated with the eye may be explicitly modeled as a function of the eye's position relative to the sensors. Determining the eye position may be a matter of solving for eye position parameters (the inputs to the model) given a set of observed sensor values (the output of the model). Given knowledge of the layout and composition of magnetic articles associated with the eye, along with knowledge of the sensor geometry and sensitivity, it has been found possible to simulate what sensor outputs one would expect to correspond to any given eye position. This is done by computing the superposition of the magnetic field vectors produced by each magnetic element at all positions where there is a sensor, and then simulating the sensors output given that magnetic field vector. Depending on the particular sensor/magnetic element geometry, the equations used to simulate sensor outputs may be algebraically inverted, or approximately inverted, resulting in a set of equations that may produce the eye position, given a set of sensor outputs. The eye position may also be determined by iteratively searching the set of possible eye position values until the difference between the simulated prediction and observed sensor measurements is small. Such a search could be performed by a variety of methods well known in the art (e.g. the Nelder-Mead method, Gauss-Newton non-linear least squares, etc).

Mechanisms of transmitting a signal from a sensing device to an assistive device, computer, or machine are well known in the art, and are described, for example, in U.S. Pat. No. 6,845,938, filed Sep. 19,2001, entitled “System and method for periodically adaptive guidance and control”; U.S. Pat. No. 6,847,287, filed Jun. 11, 2001, entitled “Transmitter-receiver control system for an actuator and method; U.S. Pat. No. 6,847,269, filed Nov. 15, 2001, entitled “High-frequency module and wireless communication device, each of which is incorporated herein by reference in their entirety. For example, the signal of an embodiment of the present invention may be transmitted by a circuit, a wire, a cable, or electromagnetic radiation, etc. Alternatively, the signal may use a communication protocol for transmitting data. Communication protocols for transmitting data are well known in the art, and are described, for example, in Data Communications and Networking, Forouzan B (Elizabeth Jones, 2004).

An embodiment of the present invention may include a variety of types of computers; e.g. a desktop computer, a laptop computer, a minicomputer, a mainframe computer, or a server, etc. The machine accessory to a computer of an embodiment of the present invention may be, for example, a mouse, a cursor, or a screen pointer. The machine may also be a computer application; e.g. a multimedia application, or any other type of computer, computer accessory, or computer application known in the art.

In another embodiment, the machine may be a mechanism of transportation., for example, an airplane, helicopter, glider, land vehicle, etc. In other embodiments, the machine may be a weapon, an ammunition system, a ballistic device, or an aviation device. For example, the device depicted in FIG. 1 can be used for aiming a weapon or ballistic device, or any other type of machine requiring guidance control.

An embodiment of the present invention may be used for controlling, for example, an electronically aimed head-mounted laser that points in the direction of the subject or user's gaze by data created from the changing magnetic field detected by a sensing device. The laser spot generated by this laser may be detected, in one embodiment, with a camera or other suitable optical device to determine where the subject or user is looking. Another embodiment of the present invention may be used with a freely moving subject in conjunction with a head-tracking device to recover the direction of gaze of the subject relative to an arbitrary reference frame.

A still other embodiment of the present invention may provide for a method for diagnosing or treating vision and eye-movement related medical disorders. In a further embodiment, the present invention may provide a motion capture system, including a method of the present invention. A motion capture system of an embodiment of the present invention may be used, for example, for capturing, recording, and replicating an actor's eye movements for animating computer-generated characters having realistic eye movements.

Another embodiment of the present invention may provide a method for instructing an assistive device, including detecting a magnetic field with a sensing device, whereby the magnetic field is generated by a magnetic article associated with an eye of a user of the machine and the sensing device is associated with the user; and transmitting an instruction to the machine, thereby instructing an assistive device, wherein the user may be challenged or disabled. Alternatively, the user may be a pilot, a navigator, a soldier, or any other type of user known in the art.

The method for tracking a subject's eye movement or eye position may be in a research application. The research application may be related to vision, ergonomics, an empirical application, e.g. use by an advertising agency to track the manner in which subjects view advertisements, or any other research application known in the art that relates to eye movement or position.

In another embodiment, the present invention may further include an additional sensing device. Thus, two or more sensing devices may be used for ascertaining the position of the magnetic article. Some embodiments of sensing devices that may not further include an additional sensing device are depicted in FIGS. 5e-f.

The sensing devices of an embodiment of the present invention may be arranged in an array, a matrix, or any other type of arrangement known in the art The array may be a rectangular array, a polar array, a hub-and-spoke array, or any other type of array known in the art. The sensing devices may be arranged in a matrix or array preparatory to or as part of a method of the present invention.

The devices, apparatuses, and methods of some embodiments of the present invention may be used, in conjunction with a human body, a non-human body, or an animal body.

The sensing device of an embodiment of the present invention may also be associated with the eye with the magnetic article external to the eye, instead of the reverse. For example, in one embodiment, the array of magnetoresistive sensors and any accompanying amplification, filtering and/or analog-to-digital conversion electronics may be associated with the eye and powered by a small battery also embedded in the contact lens. In another embodiment, the electronics may be powered by wireless power transmission (e.g. using a method such as Radio Frequency Identification (RFID), where the electronics of a device may be powered through electromagnetic induction by an externally generated electromagnetic field). Alternatively, data from the eye-associated sensor may be wirelessly transmitted to a recording device (e.g. using a method such as RFID, or using electromagnetic fields). Although the magnetic article in such an embodiment may be attached to the subject or user's head or body as described above for the sensing device in an embodiment of the present invention, the system may alternatively use a polarizing magnetic field (e.g, the earti's field, a CRT monitor, or the magnetic fields associated with the power distribution within a building).

The sensing device of an embodiment of the present invention may detect an alternating magnetic field generated by the magnetic article. In one embodiment, an alternating current in a coil or series of coils external to the eye may induce an alternating current in a coil of wire associated with the eye, via electromagnetic induction. The current induced in the eye-associated coil in turn may generate a magnetic field that may be sensed by the sensing device. In another embodiment, the coil may further include a diode. The induced field may include large components at harmonics of the frequency of the excitation of the external coil, aiding in distinguishing between magnetic fields maybe generated by the two coils. In another embodiment, an alternating field may be generated by an oscillator and battery associated with the eye.

### EXAMPLE

It is to be understood that the following example of the present invention is not intended to restrict the present invention since many more modifications may be made within the scope of the claims without departing from the spirit thereof.

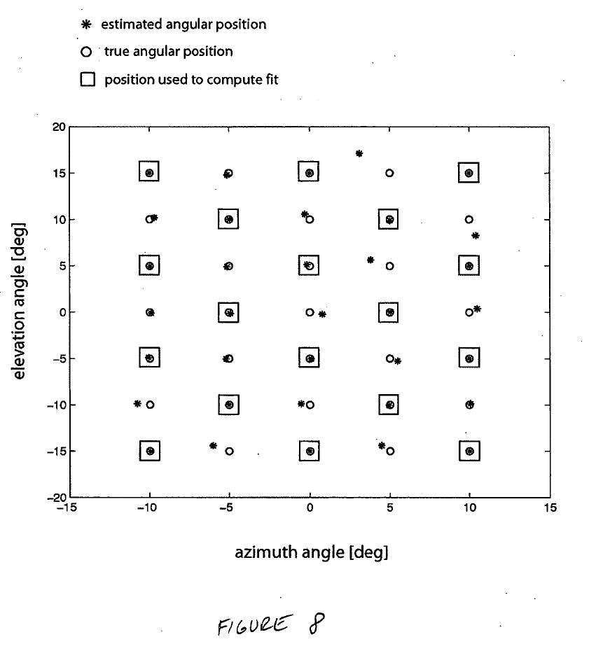

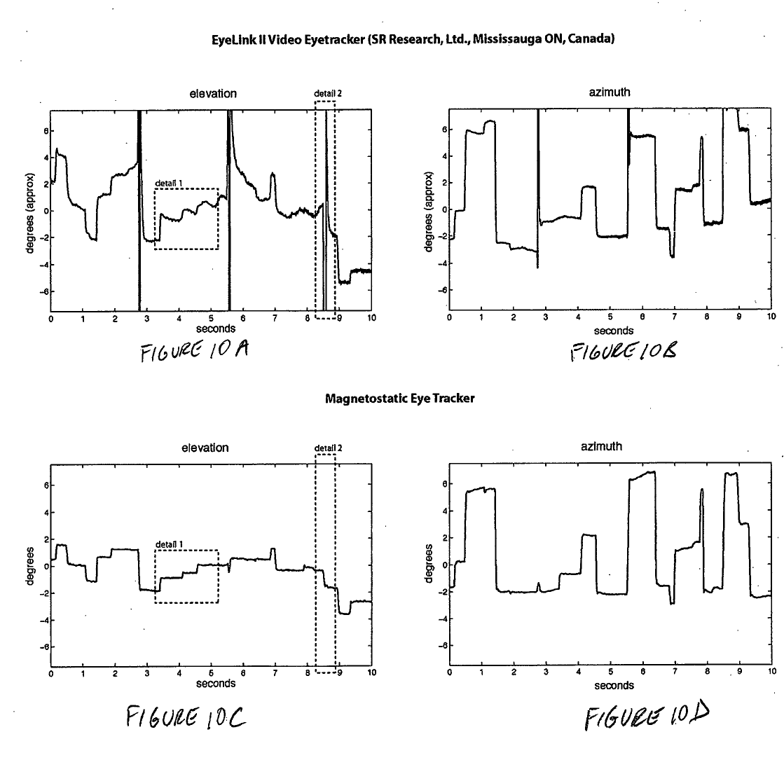

An example of an embodiment of the present invention is as follows. As illustrated generally in FIGS. 7 and 8, the output of sensors of an embodiment of the present invention was measured. Specifically, the outputs of a 3-sensor-pair embodiment of the invention was measured as a function of the systematic angular displacement of a simulated eye. A single 2 mm diameter×1 mm thick cylindrical NdFeB magnet was glued to a simulated eye. The simulated eye comprised a 2 cm diameter plastic sphere. The plastic sphere was attached to a 2-axis precision goniometer angular positioning stage. The goniometer's center-of-rotation corresponded to the sphere's center. This simulated eye apparatus allowed precise manipulation of the angle of the simulated eye to known angular positions with sub-degree accuracy. The sensors were arranged roughly as shown in FIG. 1, and with electronics as described in FIGS. 2 and 3. A National Instruments USB6009 analog-to-digital converter was used to digitize sensor voltages, and these measurements were stream to the hard disk of a personal computer. These data were analyzed offline using Matlab (Mathworks, Natick, Mass.) to produce the plots in FIGS. 10A-D, 11A-B, 12A-B, and 13A-B.

The three graphs shown in FIGS. 7A-C show the differential outputs of the sensor pairs (in volts, averaged over 0.5 seconds) of an embodiment of the invention as a function of azimuthal (horizontal) and elevation (vertical) angles of the goniometer stage. All three sensors show a smooth and consistent relationship between sensor output and the position of the simulated eye. Sensor pairs 2 and 3, in particular, show robust variations in output as a function of simulated eye position and have roughly orthogonal response surfaces.

FIG. 8 shows a subset of the measurements shown in the embodiment depicted in FIGS. 7A-C and were used to fit a function to translate sensor outputs to angular eye position. The circles in FIG. 8 show “true” angular positions of the simulated eye (measured via the vernier scales on the goniometer stage). The squares shown in FIG. 8 indicate those measurements that were used to compute the function fit. The asterisks of FIG. 8 indicate the angular positions estimated from the sensor outputs via the resulting function. The plot shows relatively good agreement between the estimated angles (asterisks) and “true” angles (circles). A polynomial function with constant, linear, interaction (e.g. x*y), and cube-root terms was fit using a linear least squares procedure.

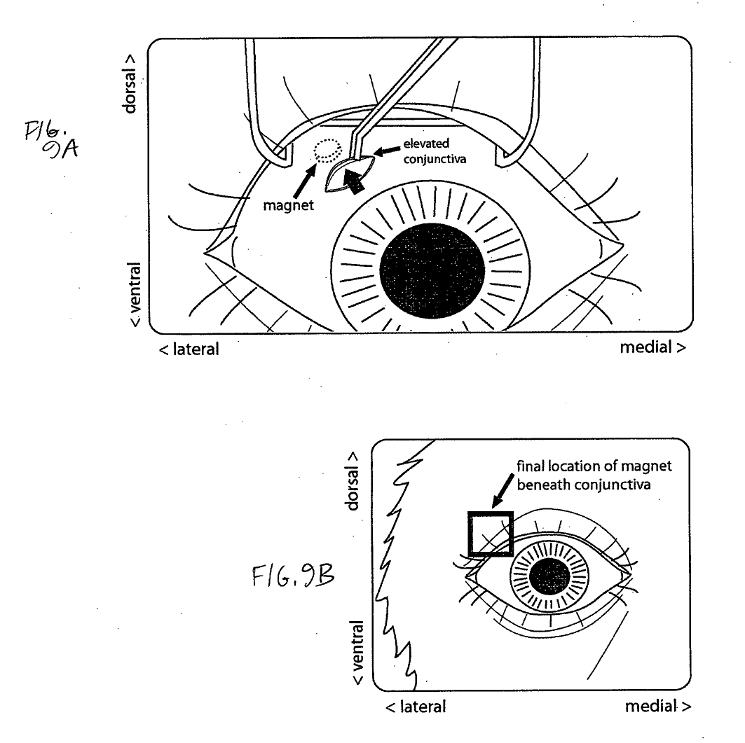

As shown in FIGS. 9A-B, an embodiment of the invention with an implanted magnetic was tested in a non-human primate (rhesus macaque monkey). All surgical procedure were conducted in compliance with the guidelines set forth by the MIT Committee on Animal Care. Under anesthesia, and using aseptic technique, a gold-plated 1 mm thick×2 mm diameter cylindrical NdFeB was implanted beneath the conjunctiva of the dorsal-lateral quadrant of the animal's right eye. As shown in FIG. 9A, a 1-2 mm incision was made in the conjunctiva and a small pouch was made just above the sclera. A magnet was placed into this pouch and the incision was closed using veterinary medical adhesive. Post-implant, there were no signs of discomfort and the animal's eye movements appeared normal. FIG. 9B shows the location of the magnet beneath the conjunctiva of the eye following implantation.

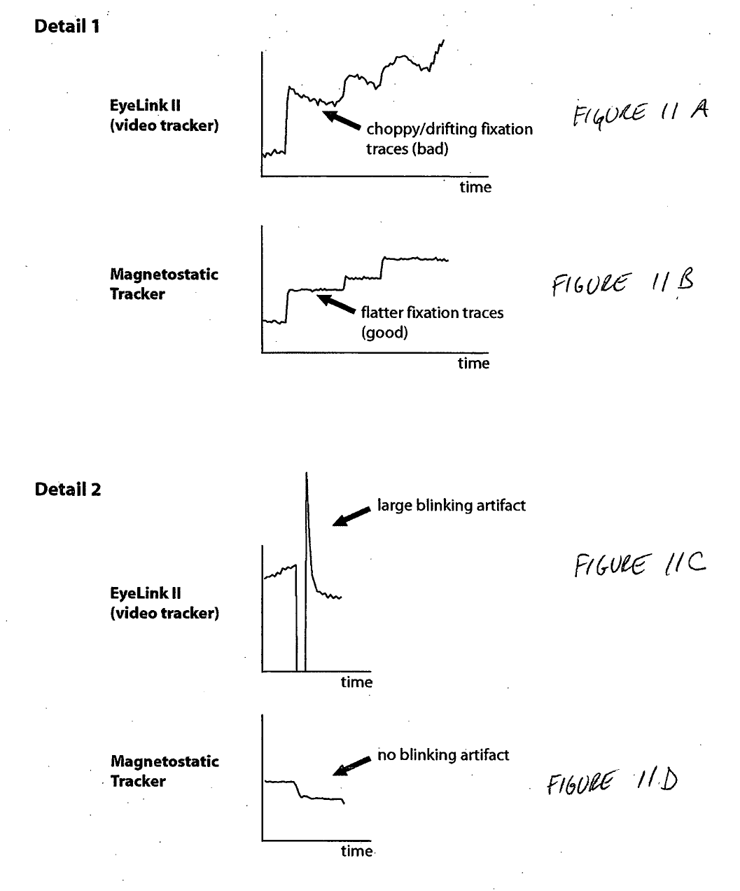

The same sensor apparatus of an embodiment described in FIGS. 7 and 8 was affixed to the animal's head such that the sensors were arranged alongside the head in analogy to the arrangement shown in FIG. 1A. FIGS. 10A-10D show sensor measurements taken with this embodiment while the animal freely moved its eyes. At the same time, an established video eyetracker (EyeLink II, SR Research, Inc. Mississauga ON, CA) was used to independently estimate the angular position of the animal's eye. Five minutes worth of data from the video eye tracker was used to fit a function translating the sensor measurements into angular eye positions (as described in FIG. 8). Subsequently, the eye position was simultaneously estimated using both the video eyetracker (FIGS. 10A and 10B) and by the embodiment of the present invention (labeled “magnetostatic eye tracker” FIGS. 10C and 10D). The plots show a typical 10 second period of estimates. Both sets of plots show all of the characteristic features of primate eye movements: rapid movements known as “saccades” punctuated by stationary periods lasting several hundreds of milliseconds, known as “fixations.” These plots show a strong correspondence between the estimates of angular position measured by the video eyetracker (FIGS. 10A and 10B) and the magnetostatic tracker (FIGS. 10C and 10D). This demonstrates that the magnetostatic tracker can in fact be used to accurately estimate angular eye position. Large transient “spikes” in the measurements taken by the video eyetracker correspond to times when the animal blinked, and the video eyetracker was unable to track the eye. (This particular embodiment of the present invention does not rely on line-of-sight, and thus does not show these spikes). Dotted-lined boxes indicate data that is further magnified in FIGS. 11A-11D.

As shown particularly in the embodiment depicted in FIGS. 11A and 11B, a magnified view of measurements shown in the graphs of FIG. 10A-D(dotted-lined box labeled “detail 1”) is illustrated. The angular eye position estimates made by an embodiment of the present invention are more stable (“flatter”) during fixations and match better with what is known about the true dynamics of saccades in humans and other primates. For example, the signal from the video eyetracker is shown as being comparatively noisier and exhibiting significant drifting during the fixations. This likely represents measurement error.

As illustrated in FIGS. 11C and 11D, simultaneous measurements were taken by both systems while the animal blinked. Because the video eyetracker relies on line-of-sight with the animal's pupil, it exhibits a large artifact when the animal blinks and the eye is occluded. In contrast, the embodiment of the present invention measured herein, does not depend on line-of-sight and continues tracking the eye, even when the eyes are closed.

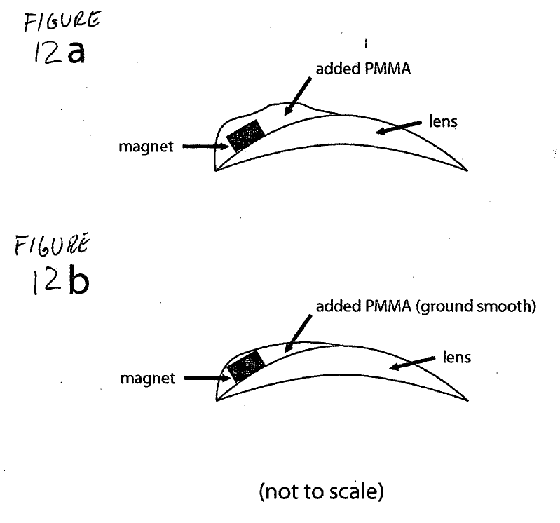

A further prophetic example of an embodiment of the present invention is as follows. A contact lens with an associated magnetic element was constructed. A schematic of this lens is shown in FIGS. 12A and 12B. A commercially available polymethyl-methacrylate (PMMA) hard contact lens was modified by placing a NdFeB cylindrical magnet (1 mm thick×2 mm diameter) onto the periphery of the outer surface of the lens and affixing it by solvent casting additional PMMA over the magnet and lens (as shown in FIG. 12A). The newly added PMMA material was molded manually while it cured and was then manually sanded smooth in order to ensure a smoothly curved, grit-free surface to the lens, as shown in FIG. 12B.

As is known to those skilled in the art, the addition of the magnetic element could also have been done during the manufacture of lens. A variety of other materials (e.g. rigid gas permeable plastics, or hydrogels) could be used, and a magnetic article could be incorporated directly into a variety of manufacture processes (e.g. molding, spin-casting, etc.) by simply incorporating the magnetic article into the plastic before it becomes solid.

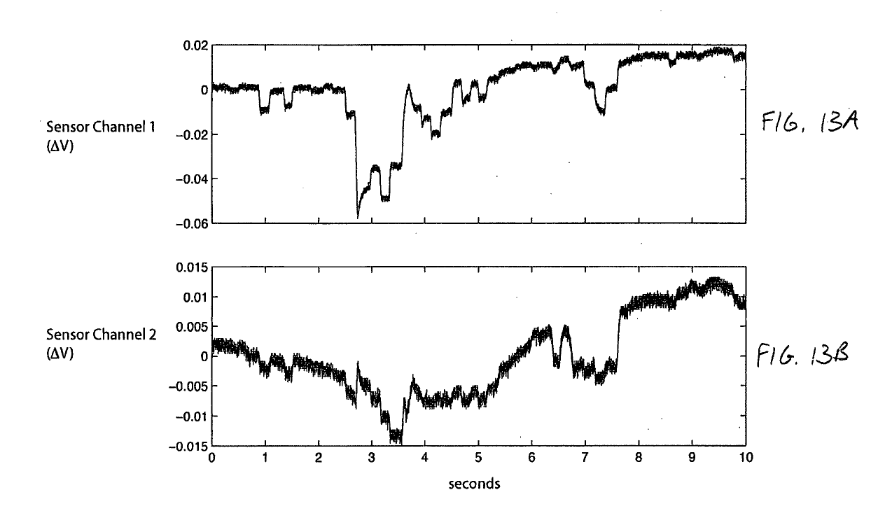

FIGS. 13A and 13B depict data collected from two sensor channels of an embodiment of the present invention where the magnetic article was attached to a contact lens (as in FIG. 12B) and the contact lens was placed in the eye of a non-human primate (a rhesus macaque monkey different from that of FIGS. 10-11). The sensor apparatus (the same as in FIGS. 10-11) was placed alongside the animal's head, next to the eye having the contact lens. The sensor outputs depict qualitatively similar data to those recorded in FIGS. 10A-D and show all of the hallmark features of eye movements, for example, rapid saccadic eye movements interspersed with fixations.

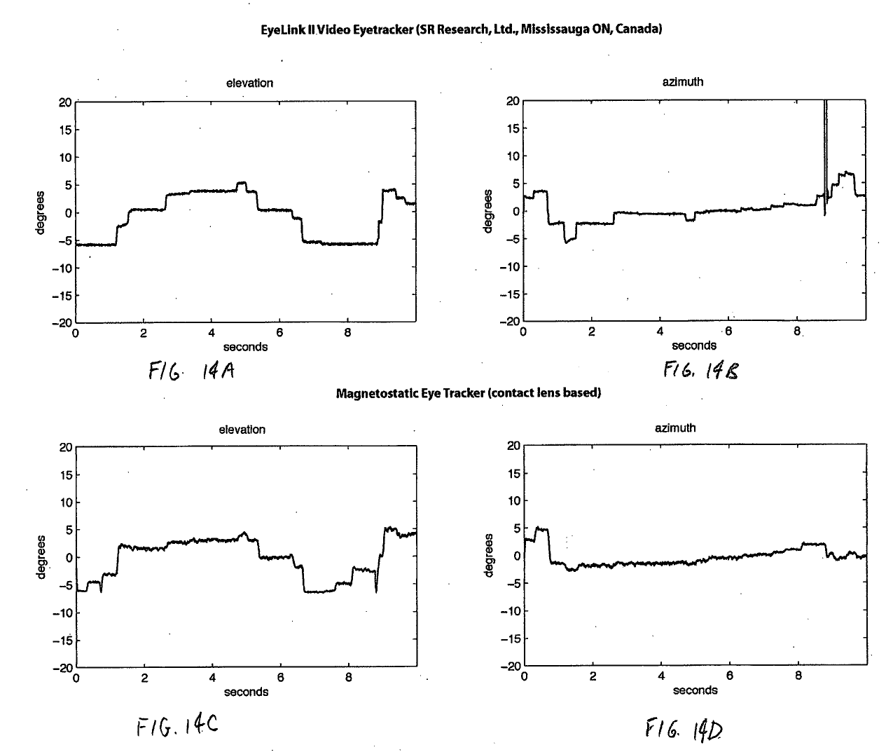

As shown in FIGS. 14A-14D, an embodiment of the invention with a magnetic embedded in a contact lens (as in FIGS. 12A-B) was tested in a non-human primate (rhesus macaque monkey). As in FIG. 10A-D, the sensor apparatus was placed alongside the animal's head. Sensor measurements were taken while the animal freely moved its eyes. At the same time, an established video eyetracker (EyeLink II, SR Research, Inc. Mississauga ON, CA) was used to independently estimate the angular position of the animal's eye. Five minutes worth of data from the video eye tracker was used to fit a function translating the sensor measurements into angular eye positions (as in FIGS. 10C-D). Subsequently, eye position was simultaneously estimated using both the video eyetracker (top plots) and by the embodiment of the present invention (labelled “magnetostatic eye tracker”). The plots show a typical 10 second period of estimates. These plots show a good correspondence between the estimates of angular position measured by the video eyetracker (top) and the magnetostatic tracker (bottom), demonstrating that the magnetostatic tracker can be used with a contact-lens-embedded magnetic article to track eye position.

Embodiments of the invention have been described for the purposes of illustration and description. The description is not intended to be exhaustive or to limit the invention to the precise form disclosed. It should be appreciated by persons skilled in the art that many modifications, variations, substitutions, changes, and equivalents are possible in light of the above teaching. It is, therefore, to be understood that the appended claims are intended to cover all such modifications and changes as fall within the true spirit of the invention.

What is claimed is:

1. An eye-tracking device for tracking movement or position of an eye of a subject, comprising:
a magnetic article adapted for association with said eye; and
a sensing device for detecting the magnetic field of said magnetic article.

2. The device of claim 1, wherein said magnetic article is associated with a contact lens for placement in said eye.

3. The device of claim 1, wherein said sensing device comprises a differential sensor, said differential sensor comprising a plurality of single-axis sensors.

4. The device of claim 1, wherein said sensing device comprises a differential sensor, said differential sensor comprising a plurality of anisotropic magnetoresistive (AMR) multi-axis sensors.

5. The device of claim 1, further comprising an assistive device and a transmitter for transmitting a signal from said sensing device to said assistive device.

6. The device of claim 5, wherein the distance between two or more of said AMR multi-axis sensors is approximately invariant.

7. The device of claim 1, wherein the directions of sensitivity of said AMR multi-axis sensors span the essentially the same linear subspace.

8. The device of claim 1, wherein said magnetic article comprises a ferromagnetic material.

9. The device of claim 1, wherein said sensing device comprises a differential sensor, said differential sensor comprising a plurality of single-axis sensors, wherein the directions of sensitivity of said single-axis sensors essentially span the same linear subspace.

10. The device of claim 9, wherein said sensing device comprises a magnetoresistive sensor.

11. The device of claim 10, wherein said magnetoresistive sensor is an anisotropic magnetoresistive (AMR) sensor.

12. The device of claim 11, further comprising a circuitry or polarizing magnet whereby the direction of sensitivity of said AMR sensor to magnetic fields can be reversed.

13. A method for tracking a movement or position of an eye of a subject, comprising:
detecting a magnetic field with a sensing device;
associating a change in the magnetic field with movement of the eye; and
transmitting data representing said eye movement.

14. The method of claim 13, further comprising inserting a contact lens having magnetic properties in the eye.

15. A method for controlling a machine by eye movement, comprising:
detecting a changing magnetic field by a sensing device associated with an eye;
creating data representing said changing magnetic field;
transmitting data representing said eye movement; and
controlling said machine by said transmitted data.

16. The method of claim 15, further comprising inserting a contact lens having magnetic properties into the eye.

17. The method of claim 15, wherein said machine is a weapon.

18. The method of claim 15, wherein said machine is a ballistic device.

19. The method of claim 15, wherein said machine is a surgical device.

20. The method of claim 15, wherein said machine is a manufacturing device.

21. A method for determining eye movement, comprising:
detecting a changing magnetic field by a sensing device associated with an eye;
accessing data associated with a changing magnetic field; and
comparing said detected field with said data for determining eye movement.

22. The method of claim 21, further comprising inserting a contact lens having magnetic properties into the eye.
